(19) United States
(12) Patent Application Publication
(10) Pub. No.: US 2024/0033468 A1
(43) Pub. Date: Feb. 1, 2024

(54) SINGLE LUMEN DRAINAGE CATHETER WITH TIP ANCHOR

(71) Applicant: Merit Medical Systems, Inc., South Jordan, UT (US)

(72) Inventor: Nicholas Accisano, III, Howell, NJ (US)

(21) Appl. No.: 18/353,502

(22) Filed: Jul. 17, 2023

Related U.S. Application Data
(60) Provisional application No. 63/369,496, filed on Jul. 26, 2022.

(51) Int. Cl. A61M 25/00

(52) U.S. Cl. CPC A61M 25/0017; A61M 25/0009

(57) ABSTRACT

A drainage catheter configured for draining bodily fluid from a patient is disclosed. The drainage catheter may comprise a catheter tube with a drainage lumen. A tension member of the drainage catheter may be disposed within the drainage lumen. The tension member may be coupled to a distal end of the catheter tube and extend proximally through a hub disposed at the proximal end of the catheter tube.

### RELATED CASES

This application claims priority to U.S. Provisional Application No. 63/369,496, filed on Jul. 26, 2022 and titled “SINGLE LUMEN DRAINAGE CATHETER WITH TIP ANCHOR,” which is hereby incorporated by reference in its entirety.

### TECHNICAL FIELD

The present disclosure relates generally to elongate medical devices, including catheters. More particularly, some embodiments relate to drainage catheters comprising a drainage lumen and a tension member.

### BRIEF DESCRIPTION OF THE DRAWINGS

FIG. 1 is a perspective view of an embodiment of a drainage catheter shown in a first configuration.

FIG. 2 is a cross-sectional end view of a catheter tube and a tension member of the drainage catheter of FIG. 1.

FIG. 3 is a detail view of a portion of a catheter hub of the drainage catheter of FIG. 1.

FIG. 4 is a cross-sectional side view of a distal portion of the drainage catheter of FIG. 1.

FIG. 5 is a perspective view of the drainage catheter of FIG. 1 shown in a second configuration.

FIG. 6A is a perspective view of a first step of a method of forming a distal tip and fixedly coupling a distal end of the tension member to the catheter tube of the drainage catheter of FIG. 1.

FIG. 6B is a cross section view of a second step of a method of forming the distal tip and fixedly coupling the distal end of the tension member to the catheter tube of the drainage catheter of FIG. 1.

FIG. 6C is a perspective view of a third step of the method of forming the distal tip and fixedly coupling the distal end of the tension member to the catheter tube of the drainage catheter of FIG. 1.

FIG. 6D is a cross section view of the third step of the method of forming the distal tip and fixedly coupling the distal end of the tension member to the catheter tube of the drainage catheter of FIG. 1.

FIG. 7A is a perspective view of a first step of another method of forming a distal tip and fixedly coupling a distal end of the tension member to the catheter tube of the drainage catheter of FIG. 1.

FIG. 7B is a cross section view of the first step of the method of forming the distal tip and fixedly coupling the distal end of the tension member to the catheter tube of the drainage catheter of FIG. 7A.

FIG. 7C is a perspective view of a second step of the method of FIG. 7A for forming the distal tip and fixedly coupling the distal end of the tension member to the catheter tube of the drainage catheter of FIG. 1.

FIG. 7D is a perspective view of the second step of the method of FIG. 7A for forming the distal tip and fixedly coupling the distal end of the tension member to the catheter tube of the drainage catheter of FIG. 1.

### DETAILED DESCRIPTION

Drainage catheters are used in a variety of medical settings for draining fluids from a patient's body. For example, a patient may suffer an injury or have a medical problem where a bodily tissue retains an excess amount of fluid, such as blood or other bodily serum. If the fluid is not removed further complications in the patient can occur such as rupturing or infection of the tissue. Accordingly, drainage catheters may be placed to remove these fluids from the patient, to either relieve pressure or otherwise ensure the fluid build-up does not result in tissue injury or other complications.

In some embodiments, drainage catheters may comprise a hub and an elongate catheter tube in fluid communication with the hub. The catheter tube may include a plurality of drainage bores. The drainage bores may be configured to allow communication of fluid from the body through the elongate catheter tube. Some drainage catheters further include a tension member or suture disposed within a portion of a drainage lumen extending along the length of the catheter tube. The tension member may be attached or secured or anchored to a catheter tip of the catheter tube to facilitate deflecting the catheter tip away from a straight configuration into a curved or pig-tail configuration to anchor the drainage catheter within a patient. For example, the practitioner may insert the catheter tube into a patient's body in a straight configuration, then pull on the tension member to form a curved loop at a distal portion of the catheter tube to anchor the drainage catheter within the body. Drainage catheters for various locations within the body, including drainage catheters for placement in the abdomen, chest, and other areas of the body are within the scope of this disclosure. Biliary and nephrostomy drainage catheters are likewise within the scope of this disclosure. Drainage catheters with drainage holes in various locations along the catheter tube, as well as drainage catheters with more than one pig-tail are also within the scope of the present disclosure.

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical and fluidic interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive). The phrase “fluid communication” is used in its ordinary sense and is broad enough to refer to arrangements in which a fluid (e.g., a gas or a liquid) can flow from one element to another element when the elements are in fluid communication with each other.

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use. With specific application to drainage catheters, the proximal end refers to the end at which the hub is disposed, while the distal end is configured for insertion into a patient when in use.

As used herein, the terms “tension member” and “suture” are broad enough to include filaments, tethers, wires, cords, straps, or other elongated flexible members configured to deflect or constrain a catheter when tension is applied to the tension member.

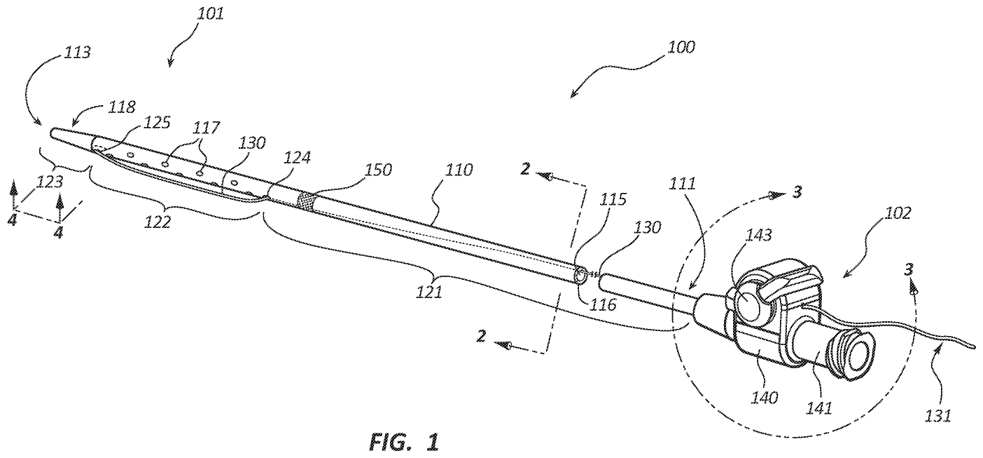

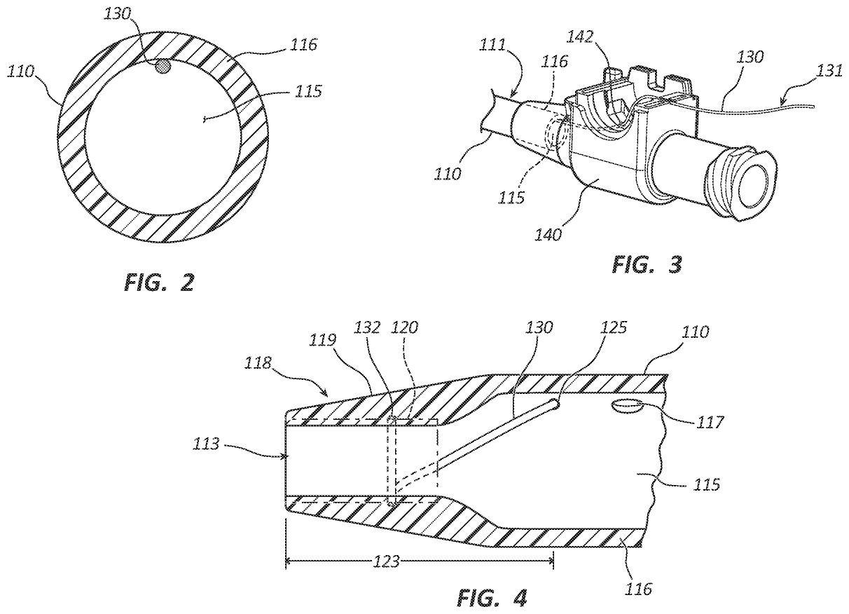

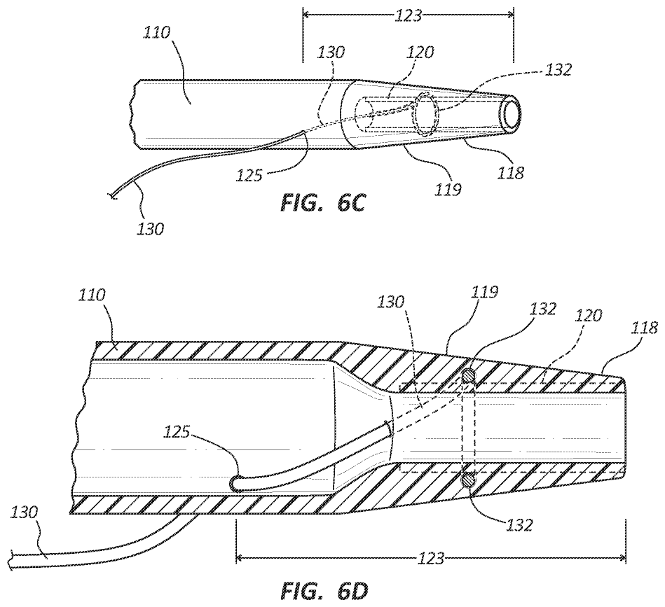

FIGS. 1-6D illustrate a drainage catheter 100 and related components. FIG. 1 is a perspective view of the drainage catheter 100 and FIG. 2 is a cross-sectional view of a catheter tube 110 and a tension member or suture 130 of the drainage catheter 100, taken through plane 2-2 of FIG. 1. The drainage catheter 100 may be used to provide a fluid pathway for draining bodily fluid from a cavity or tissue of a patient. A distal portion 101 of the drainage catheter 100 may be configured to be inserted into the patient while a proximal end 102 remains external to the patient. The drainage catheter 100 may be coupled to a fluid transfer device (not shown) to facilitate removal of excess fluid from a defined location within the patient. The distal portion 101 may be selectively disposed in a second or anchor configuration to provide for retention of the distal portion 101 within the patient as shown in FIG. 5 and further described below. Optional secondary devices such as stylets, trocars, etc. may be used in conjunction with the drainage catheter 100 to facilitate insertion and placement of the distal portion 101 within the patient. In the illustrated embodiment of FIG. 1, the drainage catheter 100 includes an elongate catheter tube 110, the tension member 130 extending along the catheter tube 110, and a catheter hub 140 coupled to a proximal end 111 of the catheter tube 110.

As illustrated in FIGS. 1 and 2, the catheter tube 110 includes a drainage lumen 115 comprising an annular wall 116 extending the length of the catheter tube 110. The catheter tube 110 may be formed of a flexible or semi-flexible bio-compatible material. For example, the catheter tube 110 may be formed of polyethylene, polypropylene, polyurethane, polyvinyl chloride, polyamide, etc. The catheter tube 110 may be sufficiently flexible to enable the drainage catheter 100 to navigate various anatomical locations within a patient. Further, the catheter tube 110 may be configured in a preformed shape in the absence of an external or internal force. The preformed shape may include straight portions and curved portions. In some embodiments the outside surface of the catheter tube 110 may be cylindrical. In other embodiments, the outside surface may have a shape other than cylindrical, for example, oval or polygonal. The drainage lumen 115 may be defined so as to maximize a cross-sectional area for fluid flow. In some embodiments, the drainage lumen 115 may comprise a circular cross section. In some embodiments, the drainage lumen 115 may be sized or otherwise configured to receive a stiffening member there through, such as a stylet or a needle.

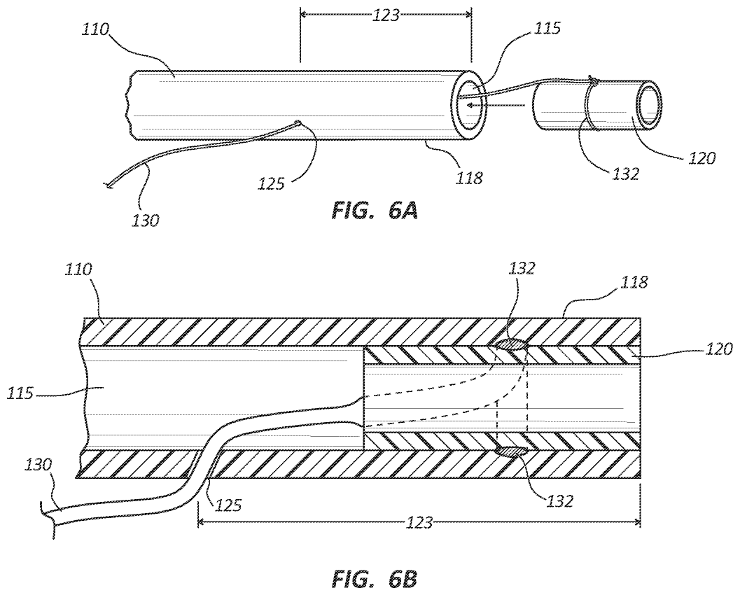

As illustrated in FIGS. 1 and 4, the catheter tube 110 may include a distal tip 118. The distal tip 118 may be shaped and sized to facilitate insertion of the distal portion 101 of the catheter tube 110 into the patient. In some embodiments, the distal tip 118 may include an external taper 119. The catheter tube 110 may include an opening at a distal end 113 of the catheter tube 110 or the distal end 113 of the catheter tube 110 may be closed.

The catheter tube 110 may comprise one or more drainage ports 117. In the illustrated embodiment of FIGS. 1 and 4, the catheter tube 110 comprises a plurality of drainage ports 117 disposed along the distal portion 101 of the catheter tube 110. Each of the drainage ports 117 comprises an orifice extending through the annular wall 116 of the catheter tube 110, providing fluid communication between an outside environment and the drainage lumen 115. The drainage ports 117 may thus be configured to provide for the passage of fluid between an anatomical location to be drained and the drainage lumen 115. The drainage ports 117 may be positioned along the catheter tube 110 such that when the distal portion 101 of the drainage catheter 100 is placed at the desired location within a patient, the drainage ports 117 are in fluid communication with the fluid to be removed from the patient.

The drainage ports 117 may be sized to facilitate fluid flow depending on the application or characteristics of a procedure. In some embodiments, the aperture size of the drainage ports 117 may be substantially similar to the drainage lumen 115. In other embodiments, the size of the drainage ports 117 may be smaller or bigger than the drainage lumen 115. The drainage ports 117 may be disposed on a single side of the catheter tube 110, or they may be distributed around the catheter tube 110. In some embodiments, the drainage ports 117 may be disposed along the inside of a curved portion of the catheter tube 110 when the distal portion 101 is disposed in the second or anchor configuration as shown in FIG. 5. Such placement may inhibit blockage of one or more drainage ports 117 by adjacent internal patient tissue.

In the illustrated embodiment of FIG. 1, the catheter hub 140 is coupled to the proximal end 111 of the catheter tube 110. The catheter hub 140 is configured to provide for fluid communication between the drainage lumen 115 and a fluid transfer device, for example, a syringe or a drainage bag. The catheter hub 140 may include a fluid connector 141 to facilitate coupling to the fluid transfer device. In some embodiments, the fluid connector 141 may be a female or male Luer connector.

For purposes of description as illustrated in FIG. 1, the catheter tube 110 may be understood as defining a proximal region 121, a central region 122, and a distal region 123. The proximal region 121 may comprise a continuous length of the catheter tube 110 extending distally from the proximal end 111 of the catheter tube 110 to an exit port 124. The exit port 124 comprises an aperture extending between the drainage lumen 115 and the exterior surface of the annular wall 116 and is configured for passage of the tension member 130 there through. In some embodiments, the exit port 124 may be one of the drainage ports 117.

The distal region 123 of the catheter tube 110 extends proximally from the distal end 113 to an entrance port 125. Similar to the exit port 124, the entrance port 125 comprises an aperture extending between the drainage lumen 115 and the exterior surface of the annular wall 116 and is configured for passage of the tension member 130 there through. In some embodiments, the entrance port 125 may be one of the drainage ports 117. The central region 122 is defined as the length segment of the catheter tube 110 extending between the exit port 124 and the entrance port 125. In the illustrated embodiment, the drainage ports 117 are shown disposed between the exit port 124 and the entrance port 125, i.e., along the central region 122. However, one or more drainage ports 117 may be disposed proximal of the exit port 124 and/or distal of the entrance port 125.

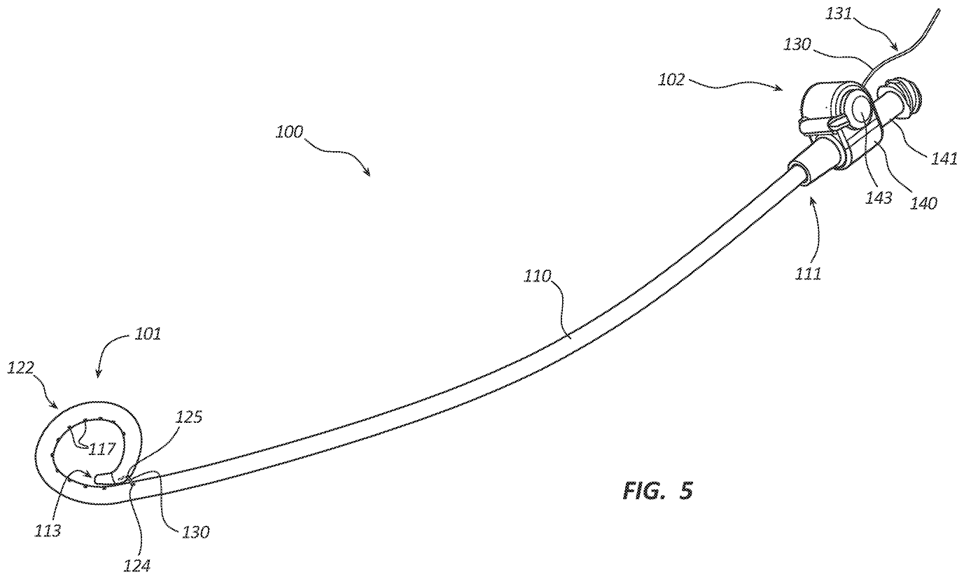

FIG. 3 is a detail view of a portion of the catheter hub 140 coupled to the proximal end 111 of the catheter tube 110. As shown in FIG. 3, the catheter hub 140 includes a tension member passageway 142 in communication with the drainage lumen 115. The tension member passageway 142 is configured for longitudinal passage of the tension member 130 through the catheter hub 140. The tension member passageway 142 may be positioned such that upon coupling of the catheter hub 140 with the catheter tube 110, the tension member passageway 142 is aligned with the drainage ports 117. As such, orientation of the drainage ports 117 can be readily determined through correlation with orientation of the catheter hub 140 when the drainage catheter 100 is inserted into a patient.

The tension member 130 may be formed of any suitable material to provide tension. In some embodiments, the tension member 130 may be of a tension member construction so as to provide tension with a minimal cross section. In some embodiments, the tension member 130 may have a guidewire formation so that the tension member 130 may be longitudinally displaced by being pushed from the proximal end 131 or the distal end 132. The tension member 130 may be formed of a thermoplastic material to accommodate the shape formation with applied energy such as heat or ultrasonic energy. The tension member 130 may comprise a material or surface treatment or coating to facilitate reduced friction within the drainage lumen 115. The tension member 130 may be formed of a biocompatible material or treated to be biocompatible. The tension member 130 may also be formed of a material compatible with bonding to the catheter tube 110.

The tension member 130 may be coupled to the catheter tube 110 such that tension on the tension member 130, or displacement of a portion of the tension member 130 relative to the catheter tube 110, may affect the shape or curvature of the catheter tube 110. For example, tension in the tension member 130 may inhibit at least a portion of the catheter tube 110 from being deflected one or more directions by an external force, for example, through contact of the catheter tube 110 with internal portions of the patient. More specifically, tension in the tension member 130 may constrain a first location of the catheter tube 110 and a second location of the catheter tube 110 to be within a defined proximity relative to each other. For example, tension on the tension member 130 may tend to pull the entrance port 125 into proximity with the exit port 124, which also results in the distal portion 101 of the catheter tube 110 assuming a curved or pigtail configuration.

As described in different terms, tension on, or displacement of, the tension member 130 relative to a portion of the catheter tube 110 may cause a curvature of at least a portion of the catheter tube 110 away from a first radius of curvature toward a second radius of curvature, wherein the second radius of curvature is less than the first radius of curvature. In some instance, the external forces acting upon the catheter tube 110 when inserted into a patient may cause a portion of the catheter tube 110 to assume a first shape. In such an instance, displacement of the tension member 130 may cause the portion of the catheter tube 110 to assume a second shape different from the first shape.

As illustrated in FIG. 1, the tension member 130 may be disposed within at least a portion of the drainage lumen 115. In the illustrated embodiment, the tension member 130 is disposed within and slidably coupled to the drainage lumen 115 along the proximal region 121 of the catheter tube 110. In some embodiments, a free proximal end 131 of the tension member 130 may extend proximally away from the catheter hub 140. The tension member 130 may exit the drainage lumen 115 through the exit port 124 and extend distally along the central region 122 external to the catheter tube 110.

The tension member 130 may re-enter the drainage lumen 115 through the entrance port 125 and be disposed within the drainage lumen 115 along at least a portion of the distal region 123. In some embodiments, the tension member 130 may be disposed within the drainage lumen 115 along the complete length or substantially along the complete length of the distal region 123. The tension member 130 may be coupled to the catheter tube 110 within the distal region 123 of the drainage lumen 115 such that displacement of the tension member 130 within the distal region 123 is inhibited. In some embodiments, a distal end 132 of the tension member 130 may be disposed within the drainage lumen 115 along the distal region 123.

Tension on, or proximal displacement of, a portion of the tension member 130 through the tension member passageway 142 may draw the entrance port 125 toward the exit port 124 and/or otherwise change the shape or curvature of the catheter tube 110. For example, proximal displacement of the tension member 130 adjacent the exit port 124 may cause deflection of the central region 122 of the catheter tube 110. In some instances, proximal displacement of the tension member 130 may cause a segment of the catheter tube 110 adjacent the entrance port 125 to contact a segment of the catheter tube 110 adjacent the exit port 124. Similarly, tension in the tension member 130 may prevent the entrance port 125 from being displaced away from the exit port 124 by a force applied to the catheter tube 110 such as, by a force applied to the distal portion 101 of the catheter tube 110. In some instances, while the drainage catheter 100 is disposed within a patient, forces may be applied to the catheter hub 140 tending to draw the drainage catheter 100 out of the patient, and thus causing internal portions of the patient to apply a reaction force to the distal portion 101 of the catheter tube 110.

As illustrated in FIG. 1, the catheter hub 140 may include a tension member lock 143. The tension member lock 143 may provide for selective prevention and allowance of displacement of the tension member 130 along the tension member passageway 142. The tension member lock 143 may be disposed in a release configuration such that upon tension applied to the tension member 130, the tension member 130 may be displaced through the tension member passageway 142. Similarly, the tension member lock 143 may be disposed in a secure configuration such that upon tension applied to the tension member 130, displacement of the tension member 130 through the tension member passageway 142 is inhibited. In some embodiments, the tension member lock 143, when in the secure configuration, may inhibit displacement of the tension member 130 through the tension member passageway 142 in a single direction, for example, in the distal direction.

FIG. 4 is a cross-sectional side view of a distal portion of the catheter tube 110. FIG. 4 shows the drainage lumen 115 extending through the distal tip 118 to the opening at the distal end 113 of the illustrated embodiment. The distal end 132 of the tension member 130 is disposed at least partially within the annular wall 116 of the distal tip 118 to secure the distal end 132 to the catheter tube 110 wherein the distal end 132 is substantially axially immovable relative to the distal tip 118. The distal tip 118 may include the external taper 119 to facilitate passage of the distal tip 118 through a patient's tissue. The distal end 132 can be disposed around an anchor member or plug 120. The materials of the catheter tube 110 and the anchor member 120 can be fused together around the distal end 132, as described below.

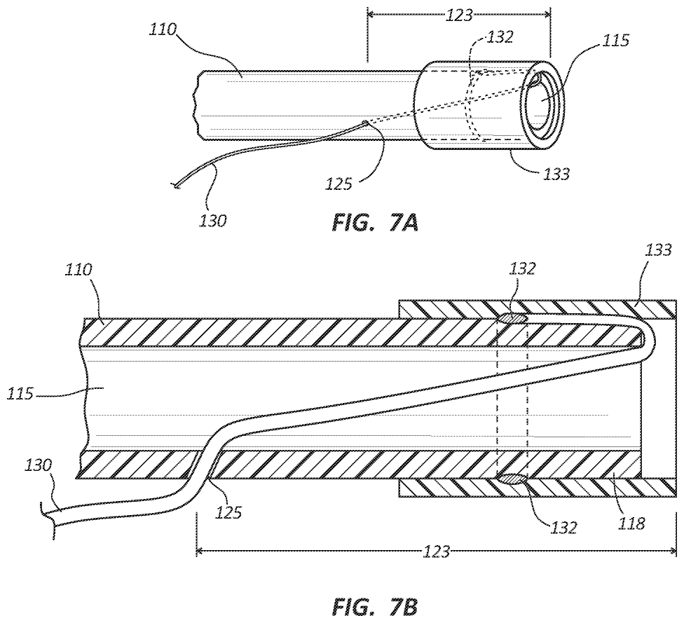

The shape of the catheter tube 110 may be altered to secure the distal end 132 of the tension member 130 to the catheter tube 110. Further, the shape of the catheter tube 110 may be altered by the application of energy applied to the external surface of the catheter tube 110, for example, heat or ultra-sonic energy. In some embodiments, the process of forming the distal tip 118 may simultaneously secure the distal end 132 of the tension member 130 to the catheter tube 110. For example, as illustrated in FIGS. 6A-6D, the distal end 132 of the tension member 130 may be coupled to an anchor member 120 by wrapping the distal end 132 around the anchor member 120. In certain embodiments, the anchor member 120 may be cylindrical and sized to be disposed within the drainage lumen 115. The distal end 132 may be wrapped around the anchor member 120 at least once and knotted to couple the tension member 130 to the anchor member 120. The tension member 130 may be disposed through the drainage lumen 115 of the distal region 123 and through the entrance port 125 as illustrated in FIGS. 6A and 6B. The anchor member 120 and the distal end 132 may be disposed within the drainage lumen 115 at the distal region 123 as illustrated in FIG. 6B. The external taper 119 of the distal tip 118 may be formed by heating and compressing the distal region 123 of the catheter tube 110 within a die and over a mandrel. This heat and compression may cause the materials of the catheter tube 110 and the anchor member 120 to melt and fuse together around the distal end 132 to secure the distal end 132 of the tension member 130 to the catheter tube 110, as illustrated in FIGS. 6C and 6D.

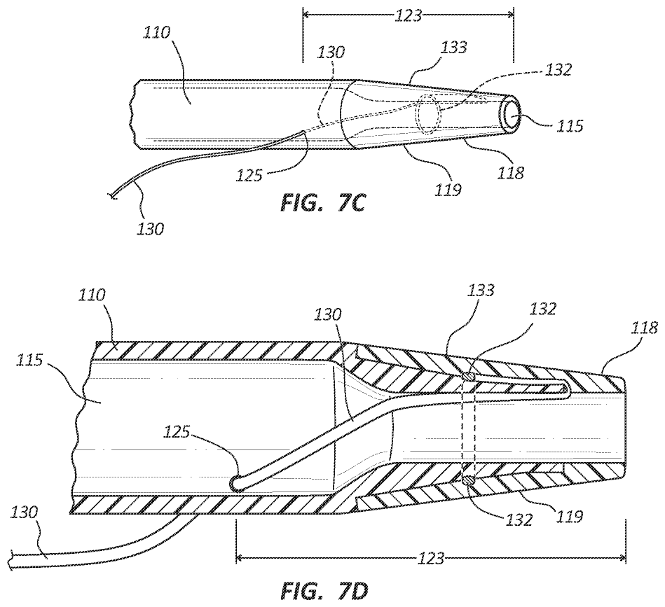

In another example, as illustrated in FIGS. 7A-7D, the distal end 132 of the tension member 130 may be coupled to the catheter tube 110 by wrapping the distal end 132 around the catheter tube 110. The distal end 132 may be wrapped around the catheter tube 110 at least once and knotted or otherwise formed into a loop to couple the tension member 130 to the catheter tube 110. The tension member 130 may be disposed through the drainage lumen 115 of the distal region 123 and through the entrance port 125 as illustrated in FIGS. 7A and 7B. In another embodiment, the tension member 130 may extend distally from the exit port 124 exterior to the catheter tube 110 and wrapped around the catheter tube 110 within the distal region 123. In other words, embodiments wherein there is no entrance port 125 and the tension member 130 extends from the exit port 124 and then is looped around the catheter tube 110 proximal of the distal end of the catheter tube 110 are within the scope of this disclosure. Whether or not the tension member 130 extends from drainage lumen 115 as shown in FIGS. 7A and 7B, or whether it is looped directly around the catheter tuber 110 after passing out of the exit port 124, an anchor member or sleeve 133 can be disposed over the distal end of the catheter tube 110 and over the distal end 132 wherein. The anchor member 133 may be cylindrical. The external taper 119 of the distal tip 118 may be formed by heating and compressing the distal region 123 of the catheter tube 110 within a die and over a mandrel. This heat and compression may cause the materials of the catheter tube 110 and the anchor member 133 to melt and fuse together around the distal end 132 to secure the distal end 132 of the tension member 130 to the catheter tube 110, as illustrated in FIGS. 7C and 7D.

With reference to the embodiment of FIGS. 6A-6D, in some embodiments, the anchor member 120 may be formed of a biodegradable material. In some such embodiments, the anchor member 120 may break down or be partially bioabsorbed during use. In some therapies, such an arrangement may facilitate removal of the drainage catheter 100. For example, in some instances, during use the tension member 130 and/or other portions of the drainage catheter 100 may become encrusted with body fluids or tissues, which may prevent the tension member 130 from moving when unlocked and thus prevent straightening of the catheter tube 110 from the curled position for removal. In embodiments wherein the anchor member 120 partially breaks down during use, it may be possible to simple pull on the proximal end of the tension member 130 to remove or partially remove the tension member 130 and, thus, unlock the drainage catheter 100 for removal. In other words, as the anchor member 120 breaks down the coupling between the tension member 130 and the distal portion of the catheter tube 110 may become weakened and facilitate this type of removal. Releasing of the coupling between the tension member 130 and the catheter tube 110 effectively unlocks the tension member 130, such that it no longer prevents straightening of the catheter tube 110 for removal.

Additionally, with respect to the embodiments of FIGS. 6A-6D and FIGS. 7A-7D, any portion of the distal end of the drainage catheter 100 may be comprised of a biodegradable or bioabsorbable material to facilitate this type of release and removal. In other words, various portions of the distal end of the assembly may be configured to break down to release the tension member 130 over time. Collagen or any other bioabsorabable or biodegradable material may be utilized in any of these embodiments.

FIG. 5 is a perspective view of the drainage catheter 100 in a second or an anchor configuration. This may be compared to the perspective view of a first or a straight configuration shown in FIG. 1. The anchor configuration may be defined as any shape or combination of shapes configured to inhibit the distal portion 101 of the drainage catheter 100 from being dislodged from the patient or withdrawn from the patient along the catheter insertion path. In some embodiments, the anchor configuration may comprise the distal region 123 of the catheter tube 110 constrained at an angle relative to the central region 122 of the catheter tube 110. In some embodiments, the anchor configuration may comprise the distal region 123 of the catheter tube 110 constrained to be within a defined distance from a central region 122 of the catheter tube 110. Tension in the tension member 130 may constrain the drainage catheter 100 in the anchor configuration and thus tend to maintain the position of the drainage catheter 100 in the anchor configuration. The tension member lock 143 may prevent distal displacement of the tension member 130 so as to constrain the drainage catheter 100 in the anchor configuration. In the illustrated embodiment, the tension member 130 is proximally displaced such that the entrance port 125 is constrained to be adjacent the exit port 124 causing a loop to be formed in the central region 122 and thus dispose the drainage catheter 100 in the anchor configuration.

As illustrated in FIG. 1, the drainage catheter 100 may include one or more radiopaque markers 150 disposed along the catheter tube 110 to facilitate visibility of the drainage catheter 100 while the drainage catheter 100 is inserted into a patient. In some embodiments, the radiopaque markers 150 may indicate the shape and location of the distal portion 101 of the catheter tube 110 and/or indicate whether or not the drainage catheter 100 is disposed in the anchor configuration.

A method of use of the drainage catheter 100 may comprise one or more of the following operational steps. The practitioner may initially dispose the tension member lock 143 in a release configuration to allow the tension member 130 to be displaced within the drainage lumen 115. With the tension member lock 143 in the release configuration, the practitioner may manipulate the distal portion 101 of the catheter tube 110 into a straight configuration, i.e., a non-anchor configuration. The practitioner may insert a stylet in the drainage lumen 115 to establish a substantially straight configuration and to provide enhanced stiffness to the catheter tube 110. In some instances, the practitioner may initially slide a cylindrical sleeve over the outside of the distal portion 101 to straighten the distal portion 101 prior to inserting a stylet or needle. The practitioner may then insert the distal portion 101 of the catheter tube 110 into the patient positioning the distal portion 101 at a desired location for the removal of fluid. The practitioner may utilize X-ray or other scanning devices to visualize one or more radiopaque markers 150 during the placement of the drainage catheter 100. The practitioner may displace the tension member 130 proximally by manually pulling on the proximal end 131 to urge the drainage catheter 100 into the anchor configuration. The tension member lock 143 may be disposed in a secure configuration to maintain tension on, and the position of, the tension member 130 to maintain the drainage catheter 100 in the anchor configuration. A fluid transfer device may be coupled to the catheter hub 140 to collect flow out of the patient. To remove the drainage catheter 100, the practitioner may dispose the tension member lock 143 into the release configuration allowing the tension member 130 to slidably displace within the drainage lumen 115 and thereby allow the distal portion 101 of the catheter tube 110 to be manipulated away from the anchor configuration. The practitioner may then draw the drainage catheter 100 out of the patient while allowing the distal tip 118 to displace the tension member 130 distally within the drainage lumen 115 as the distal portion 101 is disposed away from the anchor configuration.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Changes may be made to the details of the above-described embodiments without departing from the underlying principles of the present disclosure.

1. A drainage catheter comprising:
a catheter tube comprising a drainage lumen extending along a length of the catheter tube, the catheter tube defining a proximal region, a distal region, and a central region disposed between the proximal region and the distal region; and
a tension member disposed within the drainage lumen along the proximal region, disposed external to the drainage lumen along the central region, and disposed within the drainage lumen along at least a portion of the distal region;
wherein a distal portion of the tension member is coupled to an anchor member, and
wherein the anchor member and the distal portion of the tension member are fixedly coupled to the catheter tube.

2. The drainage catheter of claim 1, wherein the anchor member is configured to be disposed within the drainage lumen or over the catheter tube.

3. The drainage catheter of claim 1, further comprising a catheter hub coupled to a proximal end of the catheter tube, the catheter hub comprising a tension member passageway and a tension member lock, the tension member lock configured to selectively prevent and allow longitudinal displacement of the tension member along the tension member passageway.

4. The drainage catheter of claim 1, wherein the drainage lumen further comprises an exit port defining an aperture through an annular wall of the catheter tube between the drainage lumen and an external surface of the catheter tube, the exit port positioned at a distal end of the proximal region, and
wherein the tension member is disposed through the exit port.

5. The drainage catheter of claim 4, wherein the catheter tube further comprises an entrance port defining an aperture through the annular wall of the catheter tube between the drainage lumen and the external surface of the catheter tube, the entrance port positioned at a proximal end of the distal region, and
wherein the tension member is disposed through the entrance port.

6. The drainage catheter of claim 5, wherein the entrance port and the exit port are disposed in axial alignment along the catheter tube.

7. The drainage catheter of claim 1, further comprising a plurality of drainage ports disposed along the catheter tube, wherein each of the plurality of drainage ports define an aperture through an annular wall of the catheter tube between the drainage lumen and an external surface of the catheter tube.

8. The drainage catheter of claim 1, further comprising a plurality of drainage ports disposed along the central region, wherein each of the plurality of drainage ports define an aperture through an annular wall of the catheter tube between the drainage lumen and an external surface of the catheter tube.

9. The drainage catheter of claim 1, further comprising:
a plurality of drainage ports disposed along the central region, wherein each of the plurality of drainage ports define an aperture through an annular wall of the catheter tube between the drainage lumen and an external surface of the catheter tube;
an exit port defining an aperture through the annular wall of the catheter tube between the drainage lumen and the external surface of the catheter tube, the exit port positioned at a distal end of the proximal region, wherein the exit port is disposed proximal of the plurality of drainage ports, and wherein the tension member is disposed through the exit port from the drainage lumen to external of the catheter tube; and
an entrance port defining an aperture through the annular wall of the catheter tube between the drainage lumen and the external surface of the catheter tube, the entrance port positioned at a proximal end of the distal region, wherein the entrance port is disposed distal of the plurality of drainage ports, and wherein the tension member is disposed through the entrance port from external of the catheter tube into the drainage lumen.

10. The drainage catheter of claim 9, wherein the plurality of drainage ports, the exit port, and the entrance port are disposed in axial alignment along the catheter tube.

11. The drainage catheter of claim 1, further comprising:
a plurality of drainage ports disposed along the central region, wherein each of the plurality of drainage ports define an aperture through an annular wall of the catheter tube between the drainage lumen and an external surface of the catheter tube,
wherein the plurality of drainage ports comprise a proximal port and a distal port,
wherein the tension member is disposed through the proximal port from the drainage lumen to external of the catheter tube, and
wherein the tension member is disposed through the distal port from external of the catheter tube into the drainage lumen.

12. The drainage catheter of claim 1, wherein, when disposed in an anchor configuration, the central region of the catheter tube comprises a curved segment.

13. The drainage catheter of claim 12, wherein a distal tip of the catheter tube extends distally beyond the curved segment.

14. The drainage catheter of claim 1, further comprising at least one radiopaque marker disposed along the catheter tube.

15. A method of draining fluid from a patient, comprising:
obtaining a drainage catheter configured to be disposed in an anchor configuration and a non-anchor configuration, the drainage catheter comprising:
a catheter tube having a drainage lumen and a distal tip, and
a tension member disposed within at least a portion of the drainage lumen, the tension member comprising a distal end coupled to the distal tip;
inserting a distal portion of the drainage catheter into the patient to access fluid to be removed; and
disposing the drainage catheter in the anchor configuration by proximally displacing the tension member within the drainage lumen.

16. The method of claim 15, further comprising disposing a tension member lock in a secure configuration to prevent distal displacement of the distal end of the tension member.

17. The method of claim 16, further comprising disposing the tension member lock in a release configuration to allow the drainage catheter to be disposed in the non-anchor configuration by allowing distal displacement of the distal end of the tension member upon withdrawal of the drainage catheter from the patient.

18. A method of manufacturing a drainage catheter, comprising:
obtaining a catheter tube having a bore extending there through, the bore having a distal portion;
coupling a distal portion of a tension member to a distal portion of the catheter tube; and
forming a catheter tip, wherein the distal portion of the tension member is embedded in the catheter tip.

19. The method of claim 18, wherein the step of coupling the distal portion of the tension member to the distal portion of the catheter tube comprises wrapping the distal portion of the tension member around an anchor member and disposing the anchor member and the distal portion of the tension member within the distal portion of the bore.

20. The method of claim 18, wherein the step of coupling the distal portion of the tension member to the distal portion of the catheter tube comprises wrapping the distal portion of the tension member around the distal portion of the catheter tube and disposing a tubular sleeve over the distal portion of the tension member and the distal portion of the catheter tube.
